(19) United States
(12) Patent Application Publication
(10) Pub. No.: US 2024/0408158 A1
(43) Pub. Date: Dec. 12, 2024

(54) APPLICATION OF ANTARCTIC RED PIGMENT IN PREPARATION OF DRUGS FOR TREATMENT AND PREVENTION OF HYPERURICEM

(71) Applicant: LINYI UNIVERSITY, LINYI (CN)

(72) Inventors: NENGFEI WANG, LINYI (CN); SHUANG WANG, LINYI (CN); XUEMEI LI, LINYI (CN)

(21) Appl. No.: 18/501,227

(22) Filed: Nov. 3, 2023

(30) Foreign Application Priority Data
Jun. 9, 2023 (CN) 202310677580.7

(51) Int. Cl. A61K 36/06; A61P 7/00

(52) U.S. Cl. CPC A61K 36/06; A61P 7/00

(57) ABSTRACT

The present disclosure relates to an application of Antarctic red pigment in a preparation of drugs for treatment and prevention of hyperuricemia, which belongs to the field of biomedicine. The present disclosure studies a fermentation product of existing fungi Geomycos sp. wnf-18C or Geomycos sp. wnf-15A, and finds that the Antarctic red pigment produced by the fermentation product has good therapeutic effects on hyperuricemia induced by high purines. Thus, this pigment has application value in the preparation of drugs for hyperuricemia induced by high purine.

### CROSS-REFERENCE TO RELATED APPLICATIONS

This application claims priority to Chinese Patent Application No. 202310677580.7, filed on Jun. 9, 2023, which is hereby incorporated by reference in its entirety.

### TECHNICAL FIELD

The present disclosure relates to the field of biotechnology, and in particular, to an application of Antarctic red pigment in a preparation of drugs for treatment and prevention of hyperuricemia.

### BACKGROUND

Hyperuricemia is a manifestation of high uric acid concentration in the blood (male>7.0 mg/dL, female>6.0 mg/dL), it is mainly caused by an excessive production of uric acid in the body, or that the uric acid cannot be excreted in a timely manner. Hyperuricemia is divided into primary and secondary types. Primary hyperuricemia is often caused by genetic defects. Defects in the HGPRT gene can lead to increased uric acid production in the body. The decrease in uric acid excretion is the main cause of primary hyperuricemia. Reduced filtration of uric acid in the glomerulus, or decreased excretion and increased reabsorption of uric acid in the renal tubules can lead to an occurrence of hyperuricemia. Secondary hyperuricemia is closely related to drug use, dietary habits, etc. And the intake of high purine foods and alcohol consumption can easily induce hyperuricemia. In addition to causing gout, hyperuricemia is often associated with nonalcoholic fatty liver disease, diabetes, and cardiovascular disease. When hyperuricemia is asymptomatic or uric acid levels being within a controllable range, interventions are mainly carried out through exercise, diet control, and abstinence from alcohol. When uric acid levels are high or gout symptoms occur, medication treatment is necessary. The way of drugs work is to inhibit the production of uric acid or promote the excretion of uric acid. For example, allopurinol and febuxostat mainly inhibit uric acid production by inhibiting the activity of the key enzyme (xanthine oxidase) of uric acid production, while benzbromarone and probenecid promote uric acid excretion by inhibiting the reabsorption of uric acid through renal tubules. And these drugs often have obvious side effects when used in clinical practice, such as allergies, liver and kidney dysfunction, etc.

CN2015101171967, entitled with coloring compound of Antarctic fungal, which discloses a fungus Geomycos sp. wnf-15A (phy) isolated from Antarctic soil, this fungus can ferment to produce Antarctic red pigment; CN2019101866038, entitled with mutant strain of Geomycetes and application thereof, which discloses a mutant of strain Geomycos sp. wnf-15A (phy), the mutant produces an improved Antarctic red pigment, and the Antarctic red pigment has high color value, good coloring ability, high safety, and even has antioxidant and health functions. It can be used as a food additive in the food industry. But the medicinal value of this compound has not been discovered.

### SUMMARY

The technical problem to be solved by the present disclosure is to provide an application of Antarctic red pigment in a preparation of drugs for treatment and prevention of hyperuricemia.

The present disclosure is achieved through the following technical solutions.

An application of Antarctic red pigment in the preparation of drugs for treatment and prevention of hyperuricemia, where the Antarctic red pigment is derived from a fermentation product of fungi Geomycos sp. wnf 18C or Geomycos sp. wnf-15A.

In an embodiment of the present application, the hyperuricemia is induced by high purine and/or yeast fructose.

The present disclosure further provides a drug including the Antarctic red pigment, which is used for treating or preventing hyperuricemia induced by high purine and yeast fructose. The Antarctic red pigment is derived from a fermentation product of fungi Geomycos sp. wnf-18C or Geomycos sp. wnf-15A.

The beneficial effects of the present disclosure compared to prior art: the Antarctic red pigment of the present disclosure has good therapeutic effects on hyperuricemia induced by high purines. Thus, the pigment has application value in the preparation of drugs for hyperuricemia induced by high purines.

### BRIEF DESCRIPTION OF DRAWINGS

FIG. 1 is a bar chart of an effect of Antarctic red pigment on a survival rate of mice with hyperuricemia.

FIG. 2 is a linear graph of the effect of Antarctic red pigment on the survival rate of mice with hyperuricemia.

FIG. 3 is a graph showing the effect of Antarctic red pigment on 14-day uric acid levels in rats with hyperuricemia induced by fructose and yeast powder.

FIG. 4 is a graph showing the effect of Antarctic red pigment on 21-day uric acid levels in rats with hyperuricemia induced by fructose and yeast powder.

FIG. 5 is a graph showing the effect of Antarctic red pigment on 28-day uric acid levels in rats with hyperuricemia induced by fructose and yeast powder.

FIG. 6 is a graph showing the effect of Antarctic red pigment on 35-day uric acid levels in rats with hyperuricemia induced by fructose and yeast powder.

### DESCRIPTION OF EMBODIMENTS

The technical solution of the present disclosure will be further explained through embodiments in combination with drawings, but the protection scope of the present disclosure is not limited by any form of the embodiments.

The Antarctic red pigment used in the following examples is prepared according to the method disclosed in CN2019101866038, entitled with: mutant strain of Geomycetes and application thereof. The mutant strain of this patent is mutated on the basis of an original strain, which is disclosed in CN2015101171967, entitled with: coloring compound of Antarctic fungal, which disclosed the Antarctic red pigment.

### Deposit Information

Samples of Geomyces sp. wnf-18C and Geomyces sp. wnf-15A have been deposited with Chinese Type Culter Collection of Wuhan University, Wuhan, China on Jan. 24, 2019 having the deposit number of CCTCC No: M2019086, and Chinese Type Culter Collection of Wuhan University, Wuhan, China on Jun. 28, 2012 having the deposit number of CCTCC No: M201225, respectively under the terms of the Budapest Treaty on the International recognition of the Deposit of Microorganisms for the purpose of Patent Procedure.

### Example 1: Toxicity Experiment of Antarctic Red Pigment

1. Sample characteristics and treatment; Antarctic red pigment sample, red powder, stored at 4-8° C. in dark.

### 2. Experimental Animals and Reagents

2.1 The ICR mice used in the experiment were provided by Zhejiang Experimental Animal Center, with SPF level. The production license number for experimental animals is SCXK (Zhejiang) 2019-0002, and the use license number for experimental animals is SYXK (Zhejiang) 2019-0011. The experimental animal feed is provided by Zhejiang Experimental Animal Center. Detection environmental conditions: temperature range of 20-25° C. and a relative humidity range of 40-70%.

2.2 Experimental SD rats were provided by Zhejiang Experimental Animal Center, SPF level. The production license number for experimental animals is SCK (Zhejiang) 2019-0002, and the use license number for experimental animals is SYXK (Zhejiang) 2016-0022. The experimental animal feed is provided by Zhejiang Experimental Animal Center. Detection environmental conditions: temperature range of 20-25° C. and a relative humidity range of 40-70%.

2.3 Experimental reagent: 2-acetylaminofluorene: Sigma Aldrich Company, batch number: STBF2332V; 1,8-dihydroxyanthraquinone: Sigma Aldrich Company, batch number: WXBC4791V; sodium azide: Beijing Dingguo Biotechnology Co., Ltd., batch number: 81G10150; dexon: Chemservice Company, batch number: 285-75B; mitomycin C: Roche Company, batch number M0308A; cyclophosphamide: Sigma Aldrich Company, batch number: WXBC5093V.

### 3. Animal Experiments

3.1 Toxicity experiment on mice: 20 healthy and mature ICR mice with weight of 18-22 g were selected, with half male and half female. Dose and administration way: setting a dose group of 10.0 g/kg BW according to the limit method. Weighing 100 g of sample and preparing 200 mL of sample solution using distilled water as the solvent. The mice were fasted (without water) for 6 hours before gavage, and were gavaged once at a volume of 20 mL/kgBW. After 2 hours, animals ate freely.

After gavage, general condition, poisoning symptoms, and mortality of the mice were observed. The observation period is 14 days, and weights of mice at the beginning and end of the experimental period were recorded; at the end of the experiment, the mice were euthanized for gross anatomical examination and the pathological changes of the mice were recorded.

3.2 Rat experiment: 20 healthy and mature SD rats with weight of 180-220 g were selected, with half male and half female. Setting a dose group of 10.0 g/kgBW according to the limit method. Weighing 100 g of sample and preparing 200 mL of sample solution using distilled water as the solvent. Rats were fasted (without water) for 16 hours before gavage, and were gavaged once at a volume of 20 mL/kgBW. After 3 hours, the animals ate freely.

After gavage, general condition, poisoning symptoms, and mortality of the rats were observed. The observation period is 14 days, and weights of rats at the beginning and end of the experimental period were recorded; at the end of the experiment, the rats were euthanized for gross anatomical examination and the pathological changes of the rats were recorded.

3.3 Mammalian red blood cell micronucleus test: 50 healthy, mature mice with weight of 25-30 g were selected, with half male and half female. The mice were randomly divided into 5 groups, 10 mice in each group, and half male and half female in each group. The experiment was conducted with three dose groups of 1.25, 2.5, and 5.0 g/kgBW Samples of 2.5, 5.0, and 10.0 g were weighed and prepared into 40 mL sample solutions using distilled water as the solvent. Setting up another negative control group (distilled water) and a positive control group (cyclophosphamide 40 mg/kg BW, weighing 80 mg of cyclophosphamide, dissolving in sterile physiological saline to 40 mL for later use). Mice were given test substance by oral gavage at a volume of 20 mL/kgBW, gavaged twice with an interval of 24 hours. Six hours after the second gavage, the animals were euthanized due to cervical dislocation. Sternal bone marrow was taken and made into bone marrow slices, fixed with methanol, and stained with Giemsa.

Observation indicators: at least 200 red blood cells were observed in the bone marrow of each animal, and a proportion of polychromatic erythrocytes (PCE) in total red blood cells (RBC) was counted. 2000 polychromatic erythrocytes in each animal were observed and a frequency of micronucleus polychromatic erythrocytes (namely, micronucleus frequency) was counted, expressed in thousandths. Mean and standard deviation of micronucleus frequency in each group based on animal gender were calculated, Poisson distribution was used to compare micronucleus frequency in each dose group and negative control group in the test sample for the results.

### 4. Experimental Results

Acute oral toxicity test: during the experiment, there were no obvious poisoning symptoms or mortality in all rats and mice; there were no pathological changes in the organs of the rats and mice after gross dissection. The Antarctic red pigment sample (dry powder) has an oral LD50 of more than 10.0 g/kgBW for both male and female rats and mice, which indicates that it is actually non-toxic.

Mammalian red blood cell micronucleus test: PCE/RBC ratios of both male and female dose groups were not lower than 20% of that of the negative control group, and Antarctic red pigment had no cytotoxicity. Compared with the negative control group, there was no significant difference in micronucleus frequency between the male and female dose groups (P>0.05); micronucleus frequency in the positive control group was significantly higher than that of the negative control group, with a significant difference (P<0.01). Under the conditions of this experiment, there was no significant effect of Antarctic red pigment on the micronucleus frequency of mouse bone marrow red cells of mice, and the test result was negative.

### Example 2: Teratogenic Experiment of Antarctic Red Pigment

### 1.1 Test Substance: Antarctic Red Pigment Sample, Red Powder

1.2 Experimental animals: 100 SPF grade healthy female SD rats with weight of 180-220 g and 30 male rats provided by Zhejiang Experimental Animal Center were selected. The production license number for experimental animals is SCXK (Zhejiang) 2019-0002, and the use license number for experimental animals is SYXK (Zhejiang) 2016-0022. Animals adapted for 3 days before the experiment.

1.3 Animal feeding environment: room temperature of 20-25° C., relative humidity of 40-70%, feeding in stainless steel cage.

1.4 Animal feed: provided by Zhejiang Experimental Animal Center.

1.5 Test method: Oral gavage. The samples were prepared with distilled water at different concentrations according to different dose requirements. Pregnant rats were given 10 mL/kgBW by gavage once a day in the morning on the 6th to 15th day of pregnancy, while the control group was given corresponding volumes of distilled water. Setting up three dose groups of Antarctic red pigment: 0.125 g/kgBW, 0.25 g/kgBW, and 0.5 g/kgBW, and setting up another control group.

Control group: distilled water.

Low dose group: weighing 3.75 g of Antarctic red pigment, adding 300 mL of distilled water, mixing and storing in a refrigerator at 4° C., and prepared weekly.

Medium dose group: weighing 7.5 g of Antarctic red pigment, adding 300 mL of distilled water, mixing and storing in the refrigerator at 4° C., and prepared weekly.

High dose group: weighing 15 g of Antarctic red pigment, adding 300 mL of distilled water, mixing and storing in the refrigerator at 4° C., and prepared weekly.

2. Animal grouping and testing indicators: Female and male rats (in a ratio of 2:1) were kept in the same cage overnight. Those rats that were saw sperm on vaginal smears in the morning were identified as fertilized rats, and those rats became pregnant on the same day were identified as day 0. Body weight was recorded, and they were randomly divided into 4 groups, with no less than 19 rats in each group. In order to obtain sufficient fetuses to evaluate their teratogenic effects, the number of pregnant animals in each dose group should not be less than 16. Experimental rats freely ate and drank water, the general performance, behavior, poisoning symptoms, and mortality of the animals were observed and recorded. On the 6th to 15th day of conception, the test substance was orally administered daily. Weights of rats on days 0, 6, 9, 12, 15, and 20 during pregnancy were recorded. On the 20th day of pregnancy, the mother rats were euthanized, and the uterus was removed by laparotomy for weighing. The number of corpus luteum, live fetuses, absorptive fetuses, and number of stillbirths were checked and recorded. Body length, tail length, weight, and gender of live fetuses were recorded one by one, and any appearance abnormalities were checked. Half of the fetuses from each litter were immersed in 95% ethanol and fixed for 3 weeks. After rinsing, they were transparent with 2% potassium hydroxide for 3 days. After transparency, they were taken out and immersed in alizarin red for staining, and staining effect was regularly observed. Finally, skeletal abnormalities were observed; the other half fetuses were immersed in Bouins solution (Formaldehyde-Acetic acid-Picric acid solution) and fixed for 2 weeks to observe visceral malformations.

3. Data processing: Calculating the mean, standard deviation, incidence rate, etc. of each observation indicator, and conducting analysis of variance and chi-square test using PSS13.0 statistical software package.

4. Experimental results: During the experiment, there were no abnormalities in the feeding and drinking water of pregnant rats in each group, with good weight gain, no poisoning symptoms and signs, and no mortality. The effect of Antarctic red pigment on weight gain in pregnant rats refers to Table 1.

At least 19 fertilized rats were allocated to each group, and no less than 16 non fertilized rats were excluded. All indicators of pregnant rats were included in the experimental statistical range. The weight gain of pregnant rats in each dose group was good, and there was no statistically significant difference in weight, weight gain, and net gain between the pregnant rats on the 0th, 6th, 9th, 12th, 15th, and 20th days of pregnancy and the control group (P>0.05).

TABLE 1
effect of Antarctic red pigment on body weight (g) of pregnant rats (±s)

| | | | | | | | | | Weight | Net |
|---|---|---|---|---|---|---|---|---|---|---|
| | Fertilized | Pregnant | Weight (g) | | | | | | gain | gain |
| Group | rats (n) | rats (n) | 0 day | 6th day | 9th day | 12th day | 15th day | 20th day | (g) | (g) |
| Control | 19 | 16 | 242.8 ± 20.2 | 266.0 ± 22.4 | 286.8 ± 23.0 | 311.6 ± 20.8 | 340.4 ± 26.0 | 396.5 ± 27.0 | 130.5 ± 21.8 | 42.0 ± 19.0 |
| group | | | | | | | | | | |
| Low | 19 | 16 | 240.9 ± 17.8 | 272.2 ± 18.7 | 288.3 ± 21.1 | 311.7 ± 21.1 | 335.6 ± 20.7 | 399.3 ± 36.2 | 127.1 ± 24.1 | 46.1 ± 12.4 |
| dose | | | | | | | | | | |
| group | | | | | | | | | | |
| Middle | 19 | 16 | 242.3 ± 18.0 | 271.1 ± 17.6 | 288.0 ± 16.8 | 309.9 ± 21.5 | 330.0 ± 34.8 | 397.1 ± 29.7 | 126.0 ± 16.5 | 38.8 ± 13.8 |
| dose | | | | | | | | | | |
| group | | | | | | | | | | |
| High | 20 | 17 | 242.2 ± 12.7 | 273.6 ± 14.9 | 288.3 ± 13.8 | 309.0 ± 19.3 | 334.0 ± 26.7 | 407.7 ± 35.1 | 134.2 ± 27.0 | 49.6 ± 18.4 |
| dose | | | | | | | | | | |
| group | | | | | | | | | | |

The effect of Antarctic red pigment on the reproductive function of pregnant rats: the results are shown in Table 2. The implantation, absorptive fetus, live fetus, stillbirth, and mortality rate before implantation and other index of pregnant rats in each dose group were similar to those of the control group, but the differences were not statistically significant (P>0.05). Under the conditions of this experiment, no effect of Antarctic red pigment on the reproductive function of pregnant rats was observed.

TABLE 2
effects of Antarctic red pigment on reproductive function of pregnant rats

| | | | | | | | | Average | Number of |
|---|---|---|---|---|---|---|---|---|---|
| | | | | | | | | number of | mortalities |
| | Fertilized | Pregnant | Corpus | | Absorptive | | Live | live fetuses | before |
| | rats | rats | luteum | Implantation | fetus | Stillbirth | fetus | in the | implantation |
| Group | (n) | (%) | (n) | (%) | (%) | (%) | (%) | litter (±s) | (%) |
| Control | 19 | 16(84.2) | 270 | 233(86.3) | 3(1.3) | 0(0.0) | 230(98.7) | 14.4 ± 1.5 | 37(13.7) |
| group | | | | | | | | | |
| Low | 19 | 16(84.2) | 259 | 216(83.4) | 1(0.5) | 0(0.0) | 215(99.5) | 13.1 ± 3.5 | 43(16.6) |
| dose | | | | | | | | | |
| group | | | | | | | | | |
| Middle | 19 | 16(84.2) | 268 | 232(86.6) | 4(1.7) | 0(0.0) | 228(98.3) | 14.3 ± 1.7 | 36(13.4) |
| dose | | | | | | | | | |
| group | | | | | | | | | |
| High | 20 | 17(85.0) | 273 | 233(85.3) | 4(1.7) | 0(0.0) | 229(98.3) | 13.5 ± 2.7 | 40(14.7) |
| dose | | | | | | | | | |
| group | | | | | | | | | |

4.1 The effect of Antarctic red pigment on the growth and development of fetal rats: the results are shown in Table 3.

There were no statistically significant differences in body length (body length plus tail length), body weight, and gender ratio indicators between each dose group and the control group (P>0.05). Under the conditions of this experiment, there was no effect of feeding pregnant rats with Antarctic red pigment on the growth and development of fetal rats.

TABLE 3
effect of Antarctic red pigment on the growth and
development of fetal rats (±s)

| | Number | | | |
|---|---|---|---|---|
| | of | | | |
| | tested | | | |
| | fetal rats | Length | Weight | |
| Group | (n) | (cm) | (g) | Female:male |
| Control group | 230 | 5.250 ± 0.073 | 3.91 ± 0.17 | 1:1.21 |
| Low dose group | 215 | 5.292 ± 0.170 | 3.97 ± 0.46 | 1:0.95 |
| Middle dose group | 228 | 5.224 ± 0.098 | 3.80 ± 0.22 | 1:0.96 |
| High dose group | 229 | 5.240 ± 0.103 | 3.83 ± 0.22 | 1:0.88 |

4.2 The effect of Antarctic red pigment on the abnormalities of fetal rats: the results are shown in Tables 4, 5, and 6. No appearance abnormalities were observed in fetal rats for each dose group and control group. Under the conditions of this experiment, no effect of Antarctic red pigment on an occurrence of appearance abnormalities of fetal rats was observed.

TABLE 4
effect of Antarctic red pigment on the appearance abnormalities of fetal rats

| | Number of | Appearance abnormalities rate (%) | | | | |
|---|---|---|---|---|---|---|
| | tested fetal | Brain | Spina | Abdominal | Cleft | |
| Group | rats (n) | exposure | bifida | exposure | lip | Tailless |
| Control | 230 | 0(0.0) | 0(0.0) | 0(0.0) | 0(0.0) | 0(0.0) |
| group | | | | | | |
| Low | 215 | 0(0.0) | 0(0.0) | 0(0.0) | 0(0.0) | 0(0.0) |
| dose | | | | | | |
| group | | | | | | |
| Middle | 228 | 0(0.0) | 0(0.0) | 0(0.0) | 0(0.0) | 0(0.0) |
| dose | | | | | | |
| group | | | | | | |
| High | 229 | 0(0.0) | 0(0.0) | 0(0.0) | 0(0.0) | 0(0.0) |
| dose | | | | | | |
| group | | | | | | |

Absence of sternum and fontanelle enlargement of partial fetal rats in both the control group and each dose group were observed. There was no statistically significant difference (P>0.05) in absence of sternum and fontanelle enlargement between the dose groups and the control group. Under the conditions of this experiment, there was no effect of Antarctic red pigment on an occurrence of skeletal abnormalities of fetal rats.

TABLE 5
effect of Antarctic red pigment on skeletal abnormalities of fetal rats

| | | Skeletal abnormalities rate (%) | | | | | | |
|---|---|---|---|---|---|---|---|---|
| | | | | Incomplete | | | | |
| | | | | ossification | | | | |
| | | | | of posterior | | | | |
| | Number of | Absence | | head and | | | | |
| | tested fetal | of | Rib | parietal | Cervical | Sacral | Occipital | Fontanelle |
| Dose | rats (n) | sternum | deformity | bone | spondylosis | deficiency | defect | enlargement |
| Control | 120 | 103(85.8) | 0(0.0) | 0(0.0) | 0(0.0) | 0(0.0) | 0(0.0) | 0(0.0) |
| group | | | | | | | | |
| Low | 112 | 100(89.3) | 0(0.0) | 0(0.0) | 0(0.0) | 0(0.0) | 0(0.0) | 1(0.9) |
| dose | | | | | | | | |
| group | | | | | | | | |
| Middle | 118 | 112(94.9) | 0(0.0) | 0(0.0) | 0(0.0) | 0(0.0) | 0(0.0) | 1(0.9) |
| dose | | | | | | | | |
| group | | | | | | | | |
| High | 123 | 110(89.4) | 0(0.0) | 0(0.0) | 0(0.0) | 0(0.0) | 0(0.0) | 0(0.0) |
| dose | | | | | | | | |
| group | | | | | | | | |

No visceral malformations were observed in the control group and all dose groups, and under the conditions of this experiment, no effect of Antarctic red pigment on an occurrence of visceral malformations of fetal rats was observed.

TABLE 6
effect of Antarctic red pigment on visceral malformations of fetal rats

| | Number | | Visceral | | |
|---|---|---|---|---|---|
| | of | | malformation | | |
| | tested | | rate (%) | | |
| | fetal rats | Nasal | Renal | Ventricular | Cleft |
| Dose | (n) | congestion | atrophy | congestion | palate |
| Control group | 110 | 0(0.0) | 0(0.0) | 0(0.0) | 0(0.0) |
| Low dose group | 103 | 0(0.0) | 0(0.0) | 0(0.0) | 0(0.0) |
| Middle dose group | 110 | 0(0.0) | 0(0.0) | 0(0.0) | 0(0.0) |
| High dose group | 106 | 0(0.0) | 0(0.0) | 0(0.0) | 0(0.0) |

5. Conclusion: The teratogenic experiment of Antarctic red pigment on rats was conducted with three dosage groups: 0.125 g/kgBW, 0.25 g/kgBW, and 0.5 g/kgBW. No poisoning signs were observed in pregnant rats, and there are no significant detrimental effects of Antarctic red pigment on their weight gain, conception rate, live birth rate, absorptive fetus, stillbirth rate, mortality rate before implementation, and average number of live births in the litter. There were no statistically significant differences in the body weight, length, gender ratio of fetal rats between each dose group and the control group (P>0.05). In this experiment, no appearance abnormalities or visceral malformations were observed in the fetal rats. There were no statistically significant differences in absence of sternum and fontanel enlargement between the different dose groups and the control group (P>0.05). The Antarctic red pigment sample has no teratogenic effect on SD rats under the conditions of this experiment.

### Example 3: Effect of Antarctic Red Pigment on a Model of Hyperuricemia Induced by High Purine

1. Experimental animal: Balb/c male mice, 6-8 weeks old, with weigh of 16-18 g were purchased from Sperford (Beijing) Biotechnology Co., Ltd. At the Experimental Animal Center of Qingdao University, all mice were placed in pathogen free facilities, maintained at appropriate temperature and relative humidity, followed by a 12-hour light and dark cycle, they freely ate and drank, and clean bedding was replaced daily.

2. Experimental drugs: Yeast extract powder (Y820625), allopurinol (A800424), and oteracil potassium (P831461) were all purchased from McLean Biochemical Technology Co., Ltd., Antarctic red pigment.

3. Establishment a hyperuricemia model and grouping: 25 healthy Balb/c male mice were randomly divided into a blank control group, a hyperuricemia model group, a high-dose Antarctic red pigment treatment group, a low-dose Antarctic red pigment treatment group, and an allopurinol treatment group, with 5 mice in each group. Mixing Antarctic red pigment, allopurinol, and yeast extract power in a 0.5% sodium carboxymethylcellulose (0.5% CMC Na) solution. The mice in the high-dose Antarctic red pigment treatment group were given Antarctic red pigment by gavage at a weight ratio of 100 mg/kg, while the mice in the low-dose Antarctic red pigment treatment group were given Antarctic red pigment by gavage at a weight ratio of 25 mg/kg for 3 consecutive days. The other groups were not treated, and except for the blank group, the other four groups were given yeast extract suspension (0.4 g/mouse) at a weight ratio of 20 g/kg, at the same time, the high-dose Antarctic red pigment treatment group was given Antarctic red pigment by gavage at a weight ratio of 100 mg/kg, the low-dose Antarctic red pigment treatment group mice were given Antarctic red pigment by gavage at a weight ratio of 25 mg/kg, the allopurinol treatment group was given allopurinol by gavage at a weight ratio of 20 mg/kg, and the blank group was given 0.5% CMC Na by gavage at an equal volume, which was recorded as Day 1 for 7 consecutive days. From Day 5 to Day 7, except for the blank control group, the other four groups were given 0.1 ml (6 mg/animal) of oteracil potassium by intraperitoneal injection.

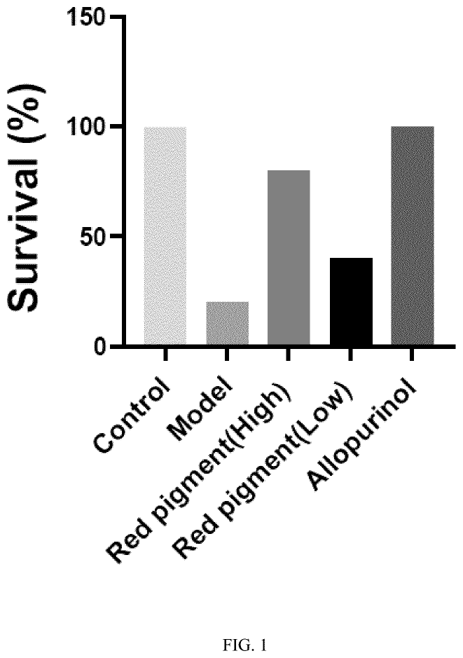

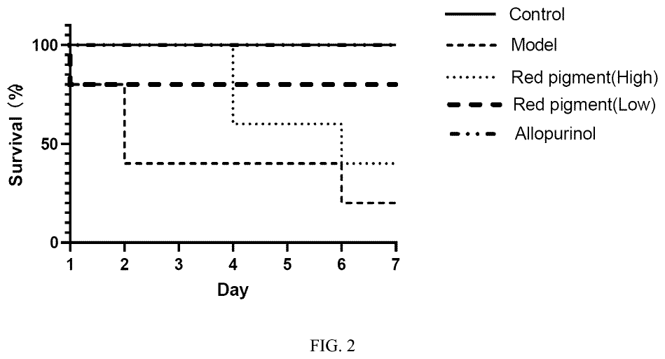

4. The results showed that there were no deaths in the blank control group and allopurinol treatment group, 4 mice in the hyperuricemia model group, 2 mice in the low-dose Antarctic red pigment treatment group, and 3 mice in the high-dose Antarctic red pigment treatment group were dead (FIG. 1). It can be seen that Antarctic red pigment treatment can significantly improve the survival rate of mice with hyperuricemia and have a certain improvement effect on hyperuricemia (FIG. 2).

### Example 4: Effect of Antarctic Red Pigment on a Model of Hyperuricemia Induced by Fructose and Yeast Diet

1. Experimental animal: Balb/c male mice, 6-8 weeks old, with weigh of 16-18 g were purchased from Sperford (Beijing) Biotechnology Co., Ltd. At the Experimental Animal Center of Qingdao University, all mice were placed in pathogen free facilities, maintained at appropriate temperature and relative humidity, followed by a 12-hour light and dark cycle, they freely ate and drank and clean bedding was replaced daily.

2. Experimental reagents and consumables: Reagents: allopurinol (A800424, MACKLIN. McLean), fructose (D809612, MACKLIN, McLean), blood glucose meter (950B, yuwell company), blood glucose test paper (591146, yuwell company), uric acid test kit (C012-2-1, Nanjing Jiancheng Biological Engineering Research Institute).

3. Method of modeling: 36 Balb/c male mice were divided into 6 groups, with 6 mice in each group. They were: Blank group (normal diet and drinking), High uric acid model group (yeast diet plus drinking water with 10% fructose), High uric acid model plus Allopurinol group (yeast diet plus drinking water with 10% fructose plus 20 mg/kg allopurinol), High uric acid model plus Low concentration pigment group (yeast diet plus drinking water with 10% fructose plus 10 mg/kg Antarctic red pigment), High uric acid model plus Medium concentration pigment group (yeast diet plus drinking water with 10% fructose plus 20 mg/kg Antarctic red pigment), High uric acid model plus High concentration pigment group (yeast diet plus drinking with 10% fructose plus 40 mg/kg Antarctic red pigment).

Dissolving allopurinol and Antarctic red pigment in double distilled water and mixing well. Starting from day 0, the Antarctic red pigment treatment group was given 10 mg/kg, 20 mg/kg, and 40 mg/kg by gavage, the Allopurinol treatment group was given 20 mg/kg by gavage, and the High uric acid model group was given an equal amount of double distilled water by gavage, once a day, for 35 consecutive days. Weights of each group of mice from day 0 onwards were recorded.

4. Blood glucose recording: At 0, 7, 14, 21, 28, and 35 days, mice were fasted for 12 hours and blood samples from the posterior orbital venous plexus of the mice at 8 pm were collected for blood glucose testing. The blood glucose values of each group of mice were recorded.

5. Uric acid testing: At Sam on days 14, 21, 28, and 35, mice were fasted for 12 hours and blood samples from the posterior orbital venous plexus of the mice were collected for uric acid testing. After the blood collection was completed, the blood was left at room temperature for one hour and centrifuged at a speed of 3000 rpm for 5 minutes to obtain a light-yellow upper serum. The serum was tested using a uric acid test kit (C012-2-1, Nanjing Jiancheng Biotechnology Research Institute), and the data were analyzed according to a formula of uric acid concentration (μmol/L)=[(A determination−A blank)/(A standard−A blank)]*C standard sample, for analysis.

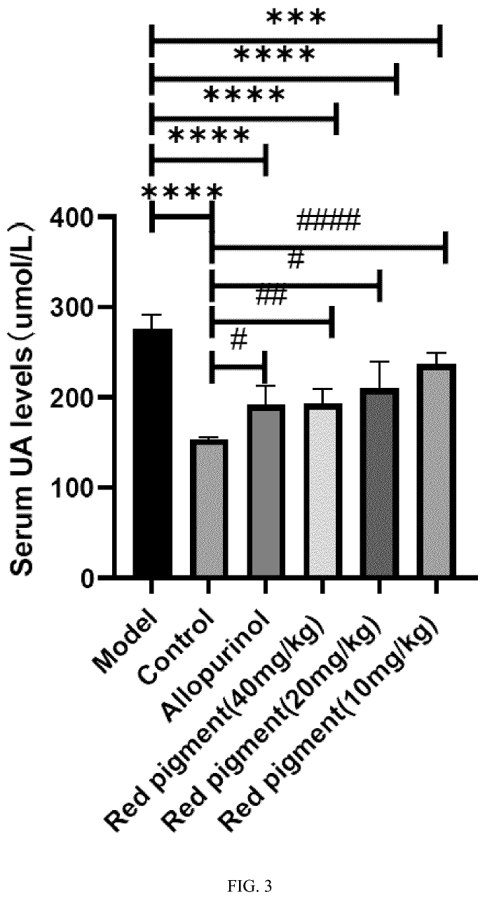

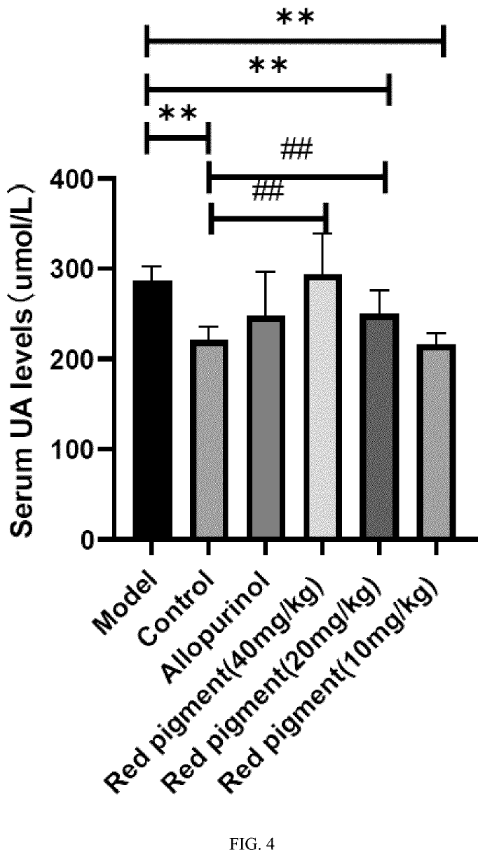

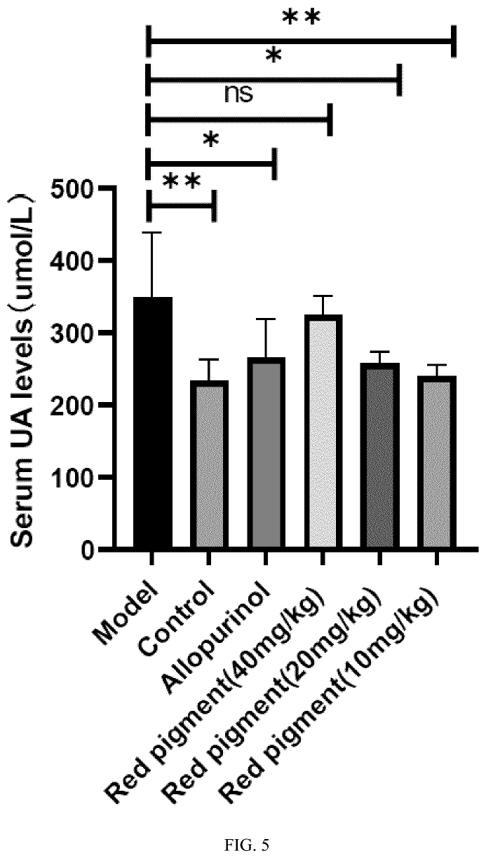

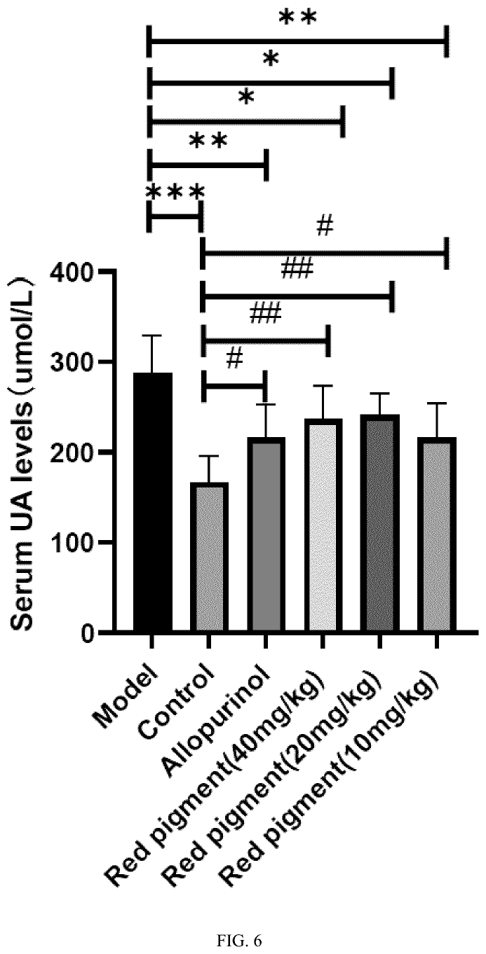

6. Result: the analysis of changes in serum uric acid levels in each group of mice (see FIGS. 3-6) showed that after 14 days of experiment, the uric acid levels in the High uric acid model group, High uric acid model plus Allopurinol group, and High uric acid model plus High/Medium/Low concentration pigment group of mice were reached to 275.1±16.18 μmol/L, 191.5±21.95 μmol/L, and 192.9±17.05/209.6±29.68/236.4±12.59 μmol/L, respectively; compared with the control group, there was a significant increase (P<0.05), with increasements of 79.10%, 24.67%, 25.59%, 36.46%, and 53.91%, and which indicates a successful modeling. The uric acid levels in the High uric acid model plus Allopurinol group and the High uric acid model plus High/Medium/Low concentration pigment group were 30.39%, 29.88%, 23.81%, and 14.07% lower than those in the High uric acid model group, respectively, and which indicates that the Antarctic red pigment has therapeutic effect.

On the 21st day, the uric acid levels in the High uric acid model plus Allopurinol group, the High uric acid model plus Medium/Low concentration pigment group were significantly reduced compared to the high uric acid model group (P<0.05), and which were reduced to 247.2±48.98 μmol/L, 249.6±27.02 μmol/L, and 215.9=13.12 μmol/L, respectively, while the uric acid level of the High concentration pigment group was increased to 293.6±45.2 μmol/L.

On the 28th day, the uric acid levels in the High uric acid model plus Allopurinol group, the High uric acid model plus High/Medium/Low concentration pigment group were significantly reduced compared to the High uric acid model group (P<0.05), which were reduced to 264.9±54.87 μmol/L, 324.8±26.57 μmol/L, 258.2±15.54 μmol/L, and 239.2 16.63 μmol/L, respectively. On the 35th day, the uric acid levels of the High uric acid model plus Allopurinol group (215.8±37.132 μmol/L), the High uric acid model plus High pigment group (236.6±37.1 gmol/L)/Medium pigment group (241.5±23.98 μmol/L)/Low pigment group (215.7±38.43 μmol/L) showed a significant decrease compared to the High uric acid model group (P<0.05); there was no significant difference in uric acid levels between the high and medium concentration pigment groups. Low concentration pigments can significantly reduce the high uric acid caused by yeast fructose.

1. A use of Antarctic red pigment in a preparation of drugs for treatment of hyperuricemia, wherein the Antarctic red pigment is derived from a fermentation product of fungi Geomyces sp. wnf-18C or Geomyces sp. wnf-15A;
wherein the hyperuricemia is induced by high purine and/or yeast fructose.

2. (canceled)
